(19) United States
(12) Patent Application Publication
(10) Pub. No.: US 2004/0127411 A1
(43) Pub. Date: Jul. 1, 2004

(54) Methods for treating lung cancer using insulin-like growth factor binding protein-3

(75) Inventor: Brian Leyland-Jones, Montreal (CA)

Correspondence Address: FOLEY AND LARDNER, SUITE 500, 3000 K STREET NW, WASHINGTON, DC, 20007, US

(73) Assignee: Insmed, Inc.

(21) Appl. No.: 10/659,708

(22) Filed: Sep. 11, 2003

Related U.S. Application Data
(60) Provisional application No. 60/409,852, filed on Sep. 11, 2002.

(51) Int. Cl. A61K 38/30

(52) U.S. Cl. 514/12

(57) ABSTRACT

The present invention relates generally to the use of Insulin like Growth Factor Binding Protein-3 (IGFBP-3) as an anti-neoplastic agent. More particularly, the invention relates to the use of IGFBP-3 in the treatment of patients with lung cancer.

This application claims the priority benefit of U.S. provisional patent application serial No. 60/409,852, filed Sep. 11, 2002.

### FIELD OF THE INVENTION

The present invention relates generally to the use of Insulin like Growth Factor Binding Protein-3 (IGFBP-3) as an anti-neoplastic agent. More particularly, the invention relates to the use of IGFBP-3 as a single agent to treat patients with lung cancer.

### BACKGROUND OF THE INVENTION

The insulin-like growth factor (IGF) system plays a pivotal role in normal growth throughout fetal and childhood development. In adult life, this system continues to function by regulating normal cellular metabolism, proliferation, differentiation and protecting against apoptotic signals. However, aberrant stimulation can contribute to the development and progression of malignant growth.

Epidemiological Evidence Implicating the IGF System in Cancer

Several years of research have demonstrated an association between excessive, aberrant signaling through the IGF-IR pathway and cancer. A number of recent epidemiological studies have suggested that reduced circulating levels of IGFBP-3, increased circulating levels of IGF-I or an increased ratio of IGF-I to IGFBP-3 are associated with an increased risk for the development of several common cancers, particularly those of the breast (Hankinson et al. 1998, Li et al. 2001), prostate (Chan et al. 2002), lung (London et al. 2002) and colon (Giovannucci et al. 2000).

IGF-I and IGF-II

IGF-I is a 70 amino acid peptide that is mainly produced by the liver in response to GH stimulation (Arany et al. 1994, Olivecrona et al. 1999), but like IGF-II, can be synthesized by almost any tissue in the body. Serum levels of IGF-I are age-dependent, increasing slowly from birth to puberty, at which point they peak and thereafter decline with age (Collett-Solberg & Cohen 2000). IGF-II is a paternally imprinted (i.e. maternally silent), 67 amino acid peptide whose serum concentration (400-600 ng/ml) is higher than IGF-I (100-200 ng/ml) at all ages, is not regulated by GH and remains stable after puberty (Moschos & Mantzoros 2002). Loss of imprinting in the IGF-II gene is often found in cancer (Jarrard et al. 1995, Oda et al. 1997, Cui et al. 1998, Kim et al. 1998, van Roozendaal et al. 1998), and most primary tumors and transformed cell lines overexpress IGF-II MRNA and protein (Werner & LeRoith 1996). The mitogenic effects of both IGFs are mediated through the IGF-IR, with growth during the embryonic and fetal stages predominantly regulated by IGF-IL and postnatally by IGF-I, which although present at lower levels, has higher affinity for the IGF-IR (Jones & Clemmons 1995). In addition to the well-established endocrine role for IGF-I, both IGFs play important paracrine and/or autocrine roles during normal development and malignant growth (see below). Liver-specific IGF-I knockout mice are viable and fertile, with a 75% reduction in circulating IGF-I levels but possessing normal tissue expression (Yakar et al. 1999). Heterozygous IGF-II gene knockout mice survive but are smaller than their wild-type littermates (DeChiara et al. 1990).

IGF Receptors and Signalling Pathways

IGFRs are cell-membrane associated glycoproteins which differ significantly in structure and function. The IGF-IR, which is expressed in most cells and resembles the insulin receptor, is a tetramer consisting of two identical extracellular α-subunits and two identical membrane-spanning β-subunits (Sepp-Lorenzino 1998). IGFs and insulin display low-affinity binding to each other's receptor (Steele-Perkins et al. 1988, Frattali & Pessin 1993), which share 60% homology. A hybrid IGF-IR/insulin receptor has been identified, which is thought to function primarily as an IGF-I receptor, since it has higher affinity for IGF-I than insulin (Jones & Clemmons 1995). The IGF-IR also binds IGF-II, but with 10-fold lower affinity than IGF-I (Rubin & Baserga 1995). The number of IGF-IRs on the cell surface is a major determinant of mitogenesis and cell survival (Rodriguez-Tarduchy et al. 1992). Malignant transformation is often associated with upregulated expression or constitutive activation of the IGF-IR (Kaleko et al. 1990, Macaulay 1992, Rubin & Baserga 1995).

IGF-Binding Proteins

IGF bioactivity is not only dependent on interaction with IGFRs, but is also influenced by the multifunctional family of IGFBPs. This superfamily includes six proteins (IGFBP-1 to IGFBP-6) that bind IGFs with high affinity and a group of IGFBP-related proteins (IGFBP-rPs 1-9) that bind IGFs with low affinity. The IGFBPs have greater affinity than the IGFRs for IGFs, and have endocrine, paracrine and autocrine effects dependent on, and independent of, IGF action. Most circulating IGFs are bound by IGFBPs, with more than 75% forming a ternary complex with IGFBP-3 (the largest and most abundant IGFBP) and the acid labile subunit (ALS). IGFBP-5 (present at levels 10% that of IGFBP-3) can also form a similar ternary complex with IGF-I or -II and ALS (Twigg & Baxter 1998), providing an additional mechanism for regulating IGF bioavailability. A small fraction of IGFs bind to IGFBPs as a binary complex, but less than 1% circulate in free form (Baxter 1994). Free or binary complexes exit the circulation rapidly, whereas ternary complexes are confined to the vascular compartment.

IGFBP-3 is also active in the cellular environment as a potent antiproliferative agent where it functions by cell cycle blockade and induction of apoptosis, independent of IGF binding. In addition to its IGF-independent effects on DNA synthesis, IGFBP-3 has been reported to directly induce apoptosis in cells lacking the IGF-IR (Rajah et al. 1997, Gill et al. 1997). This effect is at least partially attributed to IGFBP-3-induced alterations in the ratio of proapoptotic (Bax) and antiapoptotic (Bcl-2) proteins (Butt et al. 2000). IGF-independent effects are thought to be mediated by IGFBP-3 association with specific, incompletely characterized, cell surface proteins or receptors (Oh et al. 1993, Rajah et al. 1997, Leal et al. 1997, Yamanaka et al. 1999).

IGFBP-3 and Anticancer Therapy

Recent independent studies have demonstrated that IGFBP-3 can induce cell cycle arrest and enhance the efficacy of radiation, proapoptotic and chemotherapeutic agents. For example, IGFBP-3 reduces cell survival and enhances apoptosis in response to radiation in MCF-7 and T47D breast cancer cells (Butt et al. 2000, Shiry et al. 2002). Studies have also demonstrated IGFBP-3 sensitization of human retinoblastoma and gastric cancer cells to apoptosis by the topoisomerase inhibitors, etoposide, camptothecan and amsacrine (Giuliano et al. 1998, Lee et al. 2002a) and accentuation of apoptosis induced by ceramide in Hs578T human breast cancer cells (Perks et al. 2002). IGFBP-3 was shown to potentiate paclitaxel-induced cell cycle arrest and apoptosis in Hs578T human breast and gastric carcinoma cells (Fowler et al. 2000, Lee et al. 2002a). In addition, through sequestration of IGF-I, rhIGFBP-3 has been shown to restore sensitivity to Herceptin in resistant breast cancer cells expressing both the IGF-IR and HER2 (a member of the epidermal growth factor family of receptors; Lu et al. 2001).

IGFBP-3 has also been shown to have antitumor activity in vivo, either alone or in combination with standard chemotherapeutic agents. Direct injection of an IGFBP-3 expressing adenovirus into NSCLC xenografts induced destruction of tumors (Lee et al. 2002b), caused endogenous overexpression of IGFBP-3, and reduced tumor formation and/or growth of NSCLC and prostate carcinoma xenografts (Hochscheid et al. 2000, Devi et al. 2002). Furthermore, U.S. Pat. No. 5,681,818 teaches the administration of IGFBP-3 for controlling the growth of somatomedin dependent tumors in the treatment of cancer. U.S. Pat. No. 5,840,673 also describes the indirect intracellular modulation of IGFBP-3 levels as a method for controlling tumor growth. U.S. Pat. No. 6,015,786 discloses the use of IGFBP-3 complexed with mutant IGF for the treatment of IGF-dependent tumors. Collectively, these studies emphasize the value of developing IGFBP-3 as a treatment for cancer.

IGFBP-3 is no panacea for cancer, however. For example, IGFBP-3 alone is ineffective at arresting growth of breast cancer cells. (Holly, J. Biol Chem, 272:41 25602-7 (1997); Fowler et al., Int J Cancer, 88(3):448-53 (2000). Similarly, IGFBP-3 treatment does not effect oesophageal carcinomas. (Hollowood et al., Int J Cancer, 88(3):336-41 (2000) ). Thus, there remains a need to investigate the efficacy of treating cancer with IGFBP-3.

### SUMMARY OF THE INVENTION

It is, therefore, one object of the present invention to identify cancer types susceptible to insulin-like growth factor binding protein-3 (IGFBP-3) therapy.

It is also an object of the present invention to provide a method for treating lung cancer in a patient comprising administering IGFBP-3.

In accomplishing these and other objects of the invention, there is provided, in accordance with one aspect of the invention, a method for treating lung cancer in a patient consisting essentially of administering to said patient a therapeutically effective amount of insulin-like growth factor binding protein-3 (IGFBP-3).

In a preferred embodiment, the invention includes a method for treating lung cancer with IGFBP-3, wherein the treatment does not include administration of a cytotoxic and/or chemotherapeutic agent. In other preferred embodiments, IGFBP-3 is not co-administered with a cytotoxic and/or chemotherapeutic agent.

In one embodiment, the IGFBP-3 is administered parenterally. In a preferred embodiment, the IGFBP-3 is administered via intravenous infusion.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE FIGURES

FIG. 1 provides serum concentrations of IGFBP-3 in rats following IV administration of 0.8, 8, 80, and 160 mg/kg rhIGFBP-3 to rats. Data represent mean±SD.

FIG. 2 shows the pharmacokinetic profiles following SC injection of 3, 10 or 30 mg/kg rhIGFBP-3 to rats.

FIG. 3 demonstrates the growth inhibitory effect of rhIGFBP-3 combined with paclitaxel in MCF-7 breast tumor xenografts. Data represent mean±SEM; *p≦0.01 vs. control; **p≦0.05 vs. control

FIG. 4 shows the growth inhibitory effect of rhIGFBP-3 alone and in combination with irinotecan in LoVo colorectal carcinoma xenografts. Data represent mean±SEM; *p≦0.05 vs. control.

FIG. 5(A) depicts the MCF-7 breast cancer cell proliferation as assayed by MTT in increasing concentrations of rhIGFBP-3. FIG. 5(B) demonstrates the survival of MCF-7 breast cancer cells following exposure to increasing doses of radiation with rhIGFBP-3.

### DETAILED DESCRIPTION OF THE INVENTION

Definitions

As used herein and in the appended claims, the singular forms “a,” “an,” and “the” include plural reference unless the context clearly indicates otherwise. Thus, for example, reference to a “IGFBP” is a reference to one or more such proteins and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The term “protein” as used herein, includes a polymer or complex of various polymers of amino acids and does not connote a specific length of a polymer of amino acids. Thus, for example, the terms peptide, oligopeptide, and polypeptide are included within the definition of protein, whether produced using recombinant techniques, chemical or enzymatic synthesis, or naturally occurring. The term also includes peptides, oligopeptides, and polypeptides that have been modified or derivatized, such as by glycosylation, acetylation, phosphorylation, and the like. The term “protein” specifically includes variants, as defined herein.

“Insulin-like growth factor binding protein-3″ or “IGFBP-3″ as used herein is a member of the family of insulin-like growth factor binding proteins which comprises, but is not limited to, IGFBP-1, IGFBP-2, IGFBP-3, IGFBP-4, IGFBP-5, and IGFBP-6. IGFBP-3 may be obtained from natural sources or prepared by recombinant means. IGFBP-3 forms a complex with IGF and a third molecule known as ALS. Human IGFBP-3 has been cloned and sequenced. Wood W. I. et al., MOL ENDOCRINOL. (1988) 2:1176-1185. As discussed in more detail herein, the present invention contemplates variants of the IGFBP sequences. See U.S. Pat. No. 6,417,330. The term “IGFBP-3” specifically includes proteins comprising the amino sequences of the nascent full-length and the processed mature forms of IGFBP-3 as well as other IGFBP-3 thereof, derivatives and variants thereof, as defined herein. Furthermore, the term “IGFBP-3” specifically includes fusion proteins comprising IGFBP-3. All IGFBP-3 forms described herein may possess at least one of the biological activities described herein. The biological activities of the IGFBP-3 forms described herein may be assessed using the IGFBP assays described herein and otherwise known in the art. As stated herein, the term “protein” includes the term “polypeptide”. As used herein, a polypeptide includes portions of a protein including, for example, two or more amino acids. The generally preferred form of IGFBP-3 is produced recombinantly from bacteria, most preferably E. coli, consists of the native sequence of human IGFBP-3, lacks glycosylation, and does not contain chemically modified amino acids. IGFBP-3 may be expressed in any number of suitable expression systems including, for example, yeast, insect cells, mammalian cells, and bacteria. Methods for production of IGFBP-3 are known and are described, for example, in U.S. Pat. Nos. 5,200,509 and US 5,670,341 and US 6,417,330.

Assay for Biological Properties of IGFBP-3

The property of binding to an insulin-like growth factor is one of the biological activities of the IGFBPs. These proteins may be conveniently tested in a binding assay using IGF-I (Rinderknecht, E. and R. E. Humbel, J. BIOL. CHEM. (1978) 253:2769] or IGF-II [Rinderknecht, E. and R. E. Humbel, FEBS (1978) 89:283), in a labeled, e.g. iodinated form. For example, such an assay may conveniently include performing a gel electrophoresis (SDS-PAGE) of the proteins of the invention, followed by a western blot of the gel, then incubating the blot in the presence of [¹²⁵I]IGF-I or II, washing the blot to remove free IGF-I or -II, and detecting the radioactivity on the blot. IGFBP-3 binding can also be measured using SEC methodology or by isothermal calorimetry (ITC) using unlabeled IGF-I and measuring differences in chromatographic mobility (for SEC) or heats of interaction (for ITC).

“Insulin-like growth factor” or “IGF” comprises a family of protein growth factors, including, but not limited to, IGF-I and IGF-II. IGF is a polypeptide with a molecular weight of about 7.5 Kd. IGF includes naturally occurring IGF-I or IGF-II, analogs or variants thereof, and fusions between IGF-I or IGF-II and other amino acid sequences. IGF may be obtained from natural sources or prepared by recombinant means.

The term “substantially purified” refers to IGFBP-3 that may be substantially or essentially free of components that normally accompany or interact with the protein as found in its naturally occurring environment, i.e. a native cell, or host cell in the case of recombinantly produced IGFBP-3. When the IGFBP or variant thereof is recombinantly produced, the culture medium may represent less than about 30%, less than about 25%, less than about 20%, less than about 15%, less than about 10%, less than about 5%, less than about 4%, less than about 3%, less than about 2%, or less than about 1% (by dry weight) of chemical precursors or non-protein-of-interest chemicals. Thus, “substantially purified” IGFBP as produced by the methods of the present invention may have a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90% or greater as determined by appropriate methods such as SDS/PAGE analysis, RP-HPLC, SEC, capillary electrophoresis inter alia.

### METHODS OF THE INVENTION

The present invention provides methods to reduce the formation of or reduce the growth rate of a tumor at a site within the host organism. IGFBP-3 pharmaceutical formulations and dosage forms

IGFBP-3 of the invention may be administered by any conventional route suitable for proteins or peptides, including, but not limited to parenterally, e.g. injections including, but not limited to, subcutaneously or intravenously or any other form of injections or infuslions.

Pharmaceutical compositions containing the present inventive polypeptide molecule (or conjugate or fusion protein thereof) can comprise more than one active ingredient, such as more than one polypeptide molecule (or conjugate or fusion protein thereof). The pharmaceutical composition can alternatively comprise a polypeptide molecule (or conjugate or fusion protein thereof) in combination with other pharmaceutically active agents or drugs. The carrier can be any pharmaceutically acceptable suitable carrier. With respect to pharmaceutical compositions, the carrier can be any of those conventionally used and is limited only by chemico-physical considerations, such as solubility and lack of reactivity with the active compound(s), and by the route of administration. It will be appreciated by one of skill in the art that, in addition to the following described pharmaceutical compositions, the present inventive polypeptide molecule (or conjugate or fusion protein thereof) can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide molecule (or conjugate or fusion protein thereof), as well as by the particular method used to administer the polypeptide molecule (or conjugate or fusion protein thereof).

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

The polypeptide molecule (or conjugate or fusion protein thereof), alone or in combination with each other and/or with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The polypeptide molecule (or conjugate or fusion protein thereof) can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2dimethyl-1,3-dioxolane methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

A “therapeutically effective” amount of IGFBP-3 can be determined by prevention or amelioration of adverse conditions or symptoms of diseases, injuries or disorders being treated. For all the indications of use of IGFBP-3, the appropriate dosage will of course vary depending upon, for example, the tumor type and stage and severity of the disease disorder to be treated and the mode of administration. For example, tumor inhibition as a single agent may be achieved at a daily dosages from about to 0.1 mg/kg to 40 mg/kg body weight, preferably from about 0.2 mg/kg to about 20 mg/kg body weight of a binding protein of the invention. In larger mammals, for example, humans, as indicated daily dosage is from about 0.25 to about 5 mg/kg/day or about 70 mg per day for an average adult at a dose of 1 mg/kg/day conveniently administered parenterally, for example once a day.

### EXAMPLE 1

### The Pharmacokinetics of rhIGFBP-3 in Rats

Single IV Administration to Rats

The purpose of this study was to characterize the pharmacokinetic profile of rhIGFBP-3 following a single IV administration to rats. Adult male Sprague-Dawley rats (N=3/group) were administered doses of 0.8-160 mg/kg rhIGFBP-3 and blood specimens were collected for drug measurement.

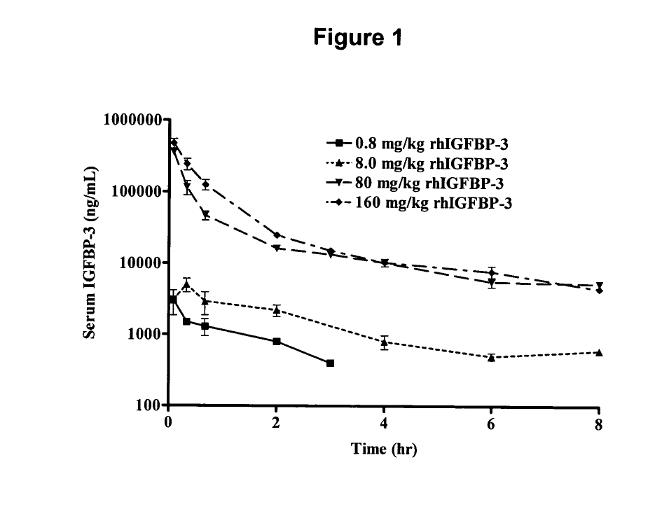

There were no adverse clinical signs associated with rhIGFBP-3 administration at any dose tested. The pharmacokinetic profiles of IGFBP-3 following increasing doses of rhIGFBP-3 are shown in FIG. 1 and Table 1.

Four hours following administration of 0.8 mg/kg rhIGFBP-3, drug concentration in the serum fell below the quantitative limitation of the assay (approximately 300 ng/mL). All other dose groups had detectable drug serum concentrations 8 hours after dosing.

The AUC and Cₘₐₓ, increased in a dose-proportional manner in the 0.8 and 80 mg/kg groups, and increased in a dose-dependent manner in the 80 and 160 mg/kg groups. The data obtained in the 8.0 mg/kg dose groups are curious, as a ten fold increase in dose produced only a 4.6 fold increase in AUC and a 1.6 fold increase in Cₘₐₓ. Although the CL/W is increased in this group, this finding can be explained by the relatively low AUC. Since there was a dose-proportional relationship between the pharmacokinetic parameters in the 0.8 and 80 mg/kg dose groups, the findings in the 8.0 mg/kg group are inexplicable.

TABLE 1
Pharmacokinetic parameters of IGFBP-3 following
IV administration of rhIGFBP-3 to rats*.

| Dose | | | |
|---|---|---|---|
| (mg/kg | AUC | Cₘₐₓ | CL/W |
| rhIGFBP-3) | (ng-hr/mL) | (ng/mL) | (mL/hr/kg) |
| 0.8 | 2172 ± 999 | 3067 ± 551 | 424 ± 187 |
| 8.0 | 10057 ± 1248 | 4967 ± 1159 | 803 ± 97 |
| 80 | 215060 ± 39102 | 360333 ± 62772 | 380 ± 64 |
| 160 | 287575 ± 64180 | 476667 ± 122202 | 579 ± 145 |

*Data represent mean ± SD.

Single SC Injection in Rats

The purpose of this study was to characterize the pharmacokinetic profile of rhIGFBP-3 following a single SC administration to rats. Adult male rats (N=5/group) were administered doses of 3-30 mg/kg rhIGFBP-3 by bolus SC injection and blood specimens were collected for drug concentration analysis.

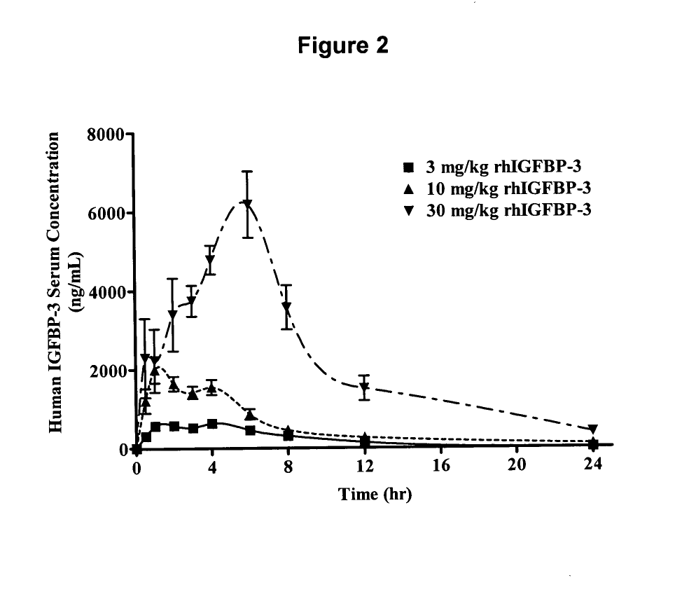

Administration of rhIGFBP-3 resulted in a dose-proportional increase in Cₘₐₓ and AUC (FIG. 2, Table 2). The AUC in the 10 and 30 mg/kg rhIGFBP-3 groups was increased by approximately 2.5 and 10-fold compared to the 3 mg/kg rhIGFBP-3 group, respectively. This study demonstrates that rhIGFBP-3 is readily absorbed into the circulation following SC administration. No signs of toxicity were noted.

TABLE 2
Cₘₐₓ and AUC for serum IGFBP-3 following
IV administration of rhIGFBP-3

| Dose and | | | |
|---|---|---|---|
| Treatment | Cₘₐₓ | AUC | CL/W |
| (mg/kg) | (ng/mL) | (ng-hr/mL) | (mL/hr/kg) |
| 3 | 751 ± 120 | 5347 ± 909 | 578 ± 122 |
| 10 | 2255 ± 217 | 12999 ± 933 | 773 ± 60 |
| 30 | 6924 ± 1021 | 54476 ± 7517 | 560 ± 85 |

### EXAMPLE 2

### Treatment of 3LL Murine Lewis Lung Tumors with rhIGFBP-3 and Carboplatin

Female C57BL6 mice (8 animals per group) received bilateral SC implants of 3LL Lewis lung tumor fragments on day 0 and were treated subcutaneously with vehicle, rhIGFBP-3 (3 or 10 mg/kg twice daily×21), carboplatin (25 or 50 mg/kg; IP; single dose) or a combination of agents beginning on day 2. Tumors were measured twice weekly for 3 weeks. rhIGFBP-3 administration did not potentiate the effects of carboplatin in this model. However, as a single agent, rhIGFBP-3 dose-dependently inhibited 3LL Lewis lung tumor growth. Table 3 shows the tumor volume upon cessation of treatment (day 23).

TABLE 3
Mean tumor volume of mice treated
with carboplatin or rhIGFBP-3.

| Treatment Group | Mean Tumor Volume (mm³) |
|---|---|
| Control | 386.13 ± 73.92 |
| Carboplatin (25 mg/kg) | 144.92 ± 31.36* |
| Carboplatin (50 mg/kg) | 187.36 ± 37.42* |
| rhIGFBP-3 (3 mg/kg twice daily) | 289.00 ± 42.97 |
| rhIGFBP-3 (10 mg/kg twice daily) | 149.73 ± 25.34* |

Data represent mean ± SEM;
*p ≦ 0.05 vs. control

The reduction in tumor volume observed in the 10 mg/kg rhIGFBP-3 group was equal to that observed with the standard chemotherapeutic agent carboplatin. Considering the toxicity associated with carboplatin therapy, in addition to the relative abundance of naturally occurring IGFBP-3 in the circulation, rhIGFBP-3 may represent a viable alternative to carboplatin in the treatment of lung cancer with substantially fewer adverse side effects.

### EXAMPLE 3

### Early Treatment of MCF-7 Human Breast Xenografts with rhIGFBP-3

Female CD1 nu/nu mice (12 animals per group) received SC implants of 5×10⁶ MCF-7 cells. On the same day, mice received either vehicle or rhIGFBP-3 IP at a dose of 20 mg/kg. Mice were then treated with either vehicle or rhIGFBP-3 (20 mg/kg twice daily) for 10 weeks. Tumor volume was monitored beginning on week 2 and measured 3 times thereafter. As shown in Table 4, rhIGFBP-3 treatment prevented MCF-7 tumor establishment compared to control. In addition, the 3 tumors that did arise in the rhIGFBP-3 group were smaller than those in the control group.

TABLE 4
Tumor parameters in control and rhIGFBP-3
treated mice after 10 weeks.

| | Number of Mice | Mean Tumor | Mean Tumor |
|---|---|---|---|
| Treatment Group | with Tumors | Volume (mm³) | Weight (g) |
| Control | 8/12 | 113.9 ± 108.9 | 0.100 ± 0.082 |
| rhIGFBP-3 | 3/12 | 37.7 ± 42.0 | 0.046 ± 0.047 |

Data represent mean ± SD.

Considering all of the mice in each group at the end of treatment, tumor volume was significantly lower in the rhIGFBP-3 treated group compared to control (p≦0.05).

### EXAMPLE 4

### Treatment of Established MCF-7 Human Breast Tumors with rhIGFBP-3 and Paclitaxel

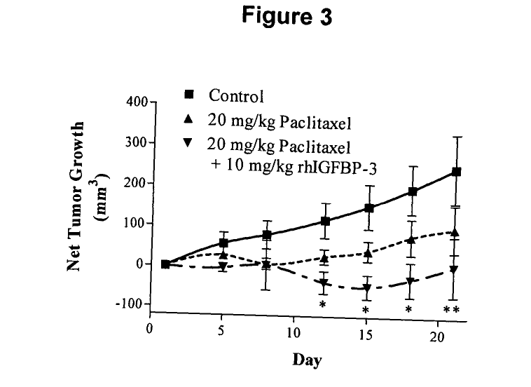

Female balb/c nu/nu mice (8 animals per group) received bilateral SC implants of MCF-7 breast tumor fragments which were allowed to grow to volumes of 100-150 mm³ prior to initiation of treatment. Upon establishment of the tumors, mice were treated with vehicle, rhIGFBP-3 (3, 10 or 30 mg/kg twice daily; SC×21), paclitaxel (10 or 20 mg/kg; IP; daily for 5 days) or a combination of agents. Tumors were measured twice weekly for 3 weeks and net tumor growth was calculated at each time point. In this model, rhIGFBP-3 did not inhibit net tumor growth when administered as a single agent. However, rhIGFBP-3 enhanced the effect of paclitaxel, with a maximum effect at doses of 10 mg/kg rhIGFBP-3 and 20 mg/kg paclitaxel (FIG. 3).

### EXAMPLE 5

### Treatment of LoVo Human Colorectal Carcinomas with rhIGFBP-3 and Irinotecan

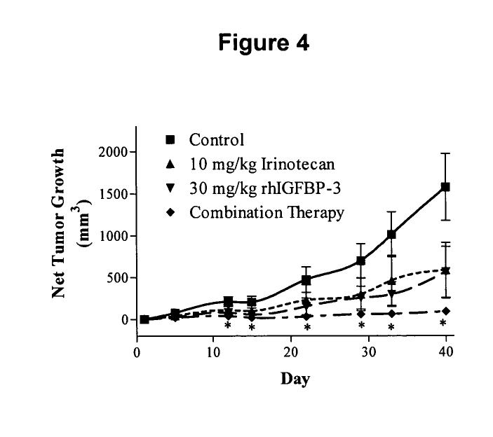

Female balb/c nu/nu mice (8 animals per group) received bilateral SC implants of LoVo colorectal carcinoma fragments which were allowed to grow to volumes of 100-150 mm3 prior to initiation of treatment. Upon establishment of the tumors, mice were treated with vehicle, rhIGFBP-3 (3, 10 or 30 mg/kg twice daily; SC x 21), irinotecan (10 or 20 mg/kg; IP; 4 doses at 4 day intervals) or a combination of agents. All treatment was discontinued on day 21 and mice were monitored for tumor response out to day 40. Net tumor growth was calculated at each time point. As depicted in FIG. 4, optimal results were obtained with a combination of 30 mg/kg rhIGFBP-3+10 mg/kg irinotecan. As a single agent, this dose of rhIGFBP-3 was equally effective in inhibiting net tumor growth as a dose of 10 mg/kg irinotecan. As all treatments were discontinued on day 21, it is important to note the persisting effect of rhIGFBP-3 and irinotecan throughout the duration of the study.

### EXAMPLE 6

### Radiosensitizing Effect of rhIGFBP-3 on MCF-7 Breast Cancer Cells in Vitro

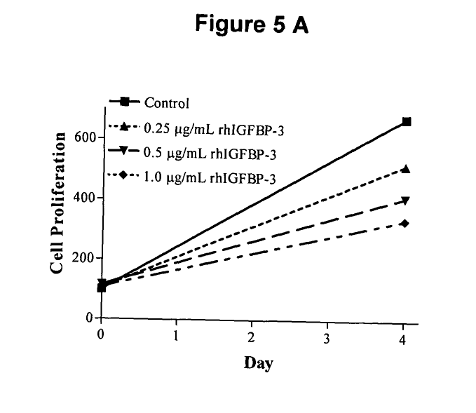

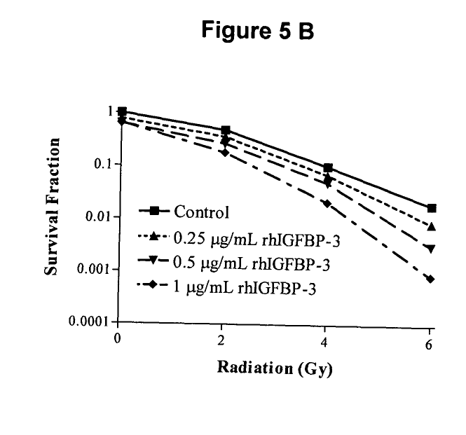

The purpose of this study was to evaluate the effects of rhIGFBP-3 alone and in combination with radiation therapy in MCF-7 human breast cancer cells. MCF-7 cells were plated and cultured in 5% FBS for 4 days with increasing concentrations of rhIGFBP-3 in the media. As shown in FIG. 5A, rhIGFBP-3 inhibited cell proliferation in a dose-dependent manner, with a maximum growth suppression of 50% compared to control values.

In a second experiment, MCF-7 cells were exposed to increasing doses of irradiation and rhIGFBP-3 24 hours after plating. Cell survival was determined on day 12. As shown in FIG. 5B, radiation alone decreased cell survival in a dose-dependent fashion. The addition of rhIGFBP-3 accentuated this effect on cell death at all doses. At a concentration of 1 μg/mL, rhIGFBP-3 decreased cell survival to ˜65%, 37%, 21% and 5% at 0, 2, 4 and 6 Gy, respectively.

### EXAMPLE 7

### Treatment of Herceptin Resistant Breast Cancer Cells with Herceptin and rhIGFBP-3

Preliminary data have shown that interference with IGF-IR signaling via co-treatment rhIGFBP-3 restores the growth-suppressive effect of Herceptin in otherwise resistant breast cancer cell lines. To further explore the ability of rhIGFBP-3 to sensitize breast tumors to Herceptin, we examined its effect in 2 HER-2-overexpressing breast cancer lines (SKBR3 and BT474) and 3 Herceptin-resistant sublines created by transfection with the IGF-IR (SKBR3/IGF-IR) or HER2 (MCF-7/HER2-18), or by prolonged exposure to Herceptin (BT474/HerR). Elevated IGF-IR levels were confirmed in all 3 Herceptin-resistant lines (Table 5). Using the MTT assay, maximal growth inhibition of parental BT474 (40%) and SKBR3 (33%) was seen at 2.5 ug/ml Herceptin. In the 3 cell lines with increased IGF-IR, survival was reduced by only 15-18% at the same Herceptin concentration. As a single agent, rhIGFBP-3 showed marked dose-dependent growth inhibition of Herceptin-resistant MCF-7/HER2-18 and SKBR3/IGF-IR, but a less pronounced effect on BT474/HerR. When combined with Herceptin, there was a marginal dose-dependent increase in growth inhibition of MCF-7/HER2-18 as compared to rhIGFBP-3 alone. In contrast, rhIGFBP-3 elicited a strong dose-dependent increase in Herceptin sensitivity of SKBR3/IGF-IR and BT474/HerR. The combination did not significantly enhance Herceptin sensitivity in BT474 and had a modest effect in SKBR3. Thus, rhIGFBP-3 displayed potent single-agent (MCF-7/HER2-18) and combinatorial activity with Herceptin (SKBR3/IGF-IR and BT474/HerR) in Herceptin-resistant breast carcinoma cells.

TABLE 5
Effect of rhIGFBP-3 and Herceptin on HER-2-overexpressing
human breast cancer cells.

| | | Growth Inhibition (MTT Assay) | | |
|---|---|---|---|---|
| | | | | Herceptin |
| Breast | | | | (2.5 μg/ml) + |
| Cancer | IGF-IR | rhIGFBP-3 | Herceptin | rhIGFBP-3 |
| Cell Line | Status | (0.1-100 μg/ml) | (2.5 μg/ml) | (0.1-100 μg/ml) |
| BT474 | Low | 10-20% | 40% | 35-47% |
| BT474/ | Mod- | 10-26% | 18% | 30-50% |
| HerR | erate | | | |
| SKBR3 | Low | 20-40% | 33% | 44-54% |
| SKBR3/ | High | 12-40% | 17% | 26-60% |
| IGF-IR | | | | |
| MCF-7/ | High | 15-55% | 15% | 20-60% |
| HER2-18 | | | | |

### REFERENCES

Arany E, Afford S, Strain A J, Winwood P J, Arthur M J & Hill D J 1994 Differential cellular synthesis of insulin-like growth factor binding protein-I (IGFBP-1) and IGFBP-3 within human liver. Journal of Clinical Endocrinology and Metabolism 79 1871-1876.

Baxter R C 1994 Insulin-like growth factor binding proteins in the human circulation: a review. Hormone Research 42 140-144.

Butt A J, Firth S M, King M A & Baxter R C 2000 Insulin-like growth factor-binding protein-3 modulates expression of Bax and Bcl-2 and potentiates p53-independent radiation-induced apoptosis in human breast cancer cells. Journal of Biological Chemistry 275 39174-39181.

Chan J M, Stampfer M J, Ma J, Gann P, Gaziano J M, Pollak M & Giovannucci E 2002 Insulin-like growth factor-I (IGF-I) and IGF binding protein-3 as predictors of advanced-stage prostate cancer. Journal of the National Cancer Institute 94 1099-1106.

Collett-Solberg P F & Cohen P 2000 Genetics, chemistry, and function of the IGF/IGFBP system. Endocrine 12 121-136.

Cui H, Horon I L, Ohlsson R, Hamilton S R & Feinberg A P 1998 Loss of imprinting in normal tissue of colorectal cancer patients with microsatellite instability. Nature Medicine 4 1276-1280.

DeChiara T M, Efstratiadis A & Robertson E J 1990 A growth-deficiency phenotype in heterozygous mice carrying an insulin-like growth factor II gene disrupted by targeting. Nature 345 78-80.

Devi G R, Sprenger C C, Plymate SR & Rosenfeld R G 2002 Insulin-like growth factor binding protein-3 induces early apoptosis in malignant prostate cancer cells and inhibits tumor formation in vivo. Prostate 51 141-152.

Fowler C A, Perks C M, Newcomb P V, Savage P B, Farndon J R & Holly J M 2000 Insulinlike growth factor binding protein-3 (IGFBP-3) potentiates paclitaxel-induced apoptosis in human breast cancer cells. International Journal of Cancer 88 448-453.

Frattali A L & Pessin J E 1993 Relationship between alpha subunit ligand occupancy and beta subunit autophosphorylation in insulin/insulin-like growth factor-i hybrid receptors. Journal of Biological Chemistry 268 7393-7400.

Gill Z P, Perks C M, Newcomb P V & Holly J M 1997 Insulin-like growth factor-binding protein (IGFBP-3) predisposes breast cancer cells to programmed cell death in a non-IGF-dependent manner. Journal of Biological Chemistry 272 25602-25607.

Giovannucci E, Pollak M N, Platz E A, Willett W C, Stampfer M J, Majeed N, Colditz G A, Speizer F E & Hankinson SE 2000 A prospective study of plasma insulin-like growth factor-1 and binding protein-3 and risk of colorectal neoplasia in women. Cancer Epidemiology, Biomarkers and Prevention 9 345-349.

Giuliano M, Lauricella M, Vassallo E, Carabillo M, Vento R & Tesoriere G 1998 Induction of apoptosis in human retinoblastoma cells by topoisomerase inhibitors. Investigative Ophthalmology and Visual Science 39 1300-1311.

Hankinson S E, Willett W C, Colditz G A, Hunter D J, Michaud D S, Deroo B, Rosner B, Speizer F E & Pollak M 1998 Circulating concentrations of insulin-like growth factor-I and risk of breast cancer. Lancet 351 1393-1396.

Hochscheid R, Jaques G & Wegmann B 2000 Transfection of human insulin-like growth factor-binding protein 3 gene inhibits cell growth and tumorigenicity: a cell culture model for lung cancer. Journal of Endocrinology 166 553-563. Jarrard D F, Bussemakers M J, Bova G S & Isaacs WB 1995 Regional loss of imprinting of the insulin-like growth factor II gene occurs in human prostate tissues. Clinical Cancer Research 1 1471-1478.

Jones J I & Clemmons D R 1995 Insulin-like growth factors and their binding proteins: biological actions. Endocrine Reviews 16 3-34.

Kaleko M, Rutter W J & Miller A D 1990 Overexpression of the human insulinlike growth factor I receptor promotes ligand-dependent neoplastic transformation. Molecular and Cellular Biology 10 464-473.

Kim H T, Choi B H, Niikawa N, Lee T S & Chang S I 1998 Frequent loss of imprinting of the H19 and IGF-II genes in ovarian tumors. American Journal of Medical Genetics 80 391-395.

Leal S M, Liu Q, Huang S S & Huang J S 1997 The type V transforming growth factor beta receptor is the putative insulin-like growth factor-binding protein 3 receptor. Journal of Biological Chemistry 272 20572-20576.

Lee D Y, Yi H K, Hwang P H & Oh Y 2002a Enhanced expression of insulin-like growth factor binding protein-3 sensitizes the growth inhibitory effect of anticancer drugs in gastric cancer cells. Biochemical and Biophysical Research Communications 294 480-486.

Lee H Y, Chun K H, Liu B, Wiehle S A, Cristiano R J, Hong W K, Cohen P & Kurie JM 2002b Insulin-like growth factor binding protein-3 inhibits the growth of non-small cell lung cancer. Cancer Research 62 3530-3537.

Li B D, Khosravi M J, Berkel H J, Diamandi A, Dayton M A, Smith M & Yu H 2001 Free insulin-like growth factor-I and breast cancer risk. International Journal of Cancer 91 736-739.

London S J, Yuan J M, Travlos G S, Gao Y T, Wilson R E, Ross R K & Yu M C 2002 Insulin-like growth factor I, IGF-binding protein 3, and lung cancer risk in a prospective study of men in China. Journal of the National Cancer Institute 94 749-754.

Lu Y, Zi X, Zhao Y, Mascarenhas D & Pollak M 2001 Insulin-like growth factor-I receptor signaling and resistance to trastuzumab (Herceptin). Journal of the National Cancer Institute 93 1852-1857.

Macaulay V M 1992 Insulin-like growth factors and cancer. British Journal of Cancer 65 311-320.

Moschos S J & Mantzoros C S 2002 The role of the IGF system in cancer: from basic to clinical studies and clinical applications. Oncology 63 317-332.

Oda H, Shimizu S, Minami K, Kaneko K & Ishikawa T 1997 Loss of imprinting of the IGF2 gene in a Wilms' tumor in an adult. Journal of the National Cancer Institute 89 1813-1814.

Oh Y, Muller H L, Pham H & Rosenfeld RG 1993 Demonstration of receptors for insulinlike growth factor binding protein-3 on Hs578T human breast cancer cells. Journal of Biological Chemistry 268 26045-26048.

Olivecrona H, Hilding A, Ekstrom C, Barle H, Nyberg B, Moller C, Delhanty P J, Baxter R C, Angelin B, Ekstrom T J & Tally M 1999 Acute and short-term effects of growth hormone on insulin-like growth factors and their binding proteins: serum levels and hepatic messenger ribonucleic acid responses in humans. Journal of Clinical Endocrinology and Metabolism 84 553-560.

Perks C M, McCaig C, Clarke J B, Clemmons D R & Holly J M 2002 A non-IGF binding mutant of IGFBP-3 modulates cell function in breast epithelial cells. Biochemical and Biophysical Research Communications 294 988-994.

Rajah R, Valentinis B & Cohen P 1997 Insulin-like growth factor (IGF)-binding protein-3 induces apoptosis and mediates the effects of transforming growth factor-betal on programmed cell death through a p53- and IGF-independent mechanism. Journal of Biological Chemistry 272 12181-12188.

Rodriguez-Tarduchy G, Collins M K, Garcia I & Lopez-Rivas A 1992 Insulin-like growth factor-I inhibits apoptosis in IL-3-dependent hemopoietic cells. Journal of Immunology 149 535-540.

Rubin R & Baserga R 1995 Insulin-like growth factor-I receptor. Its role in cell proliferation, apoptosis, and tumorigenicity. Laboratory Investigation 73 311-331.

Sepp-Lorenzino L 1998 Structure and function of the insulin-like growth factor I receptor. Breast Cancer Research and Treatment 47 235-253.

Shiry L J, Blouin M J, Lenhert S, Allan G & Pollak M 2002 Radiosensitizing effect of rhIGFBP-3 on MCF-7 breast cancer cells in vitro. Hormone Research 58 266 (abstract P20).

Steele-Perkins G, Turner J, Edman J C, Hari J, Pierce S B, Stover C, Rutter W J & Roth R A 1988 Expression and characterization of a functional human insulin-like growth factor I receptor. Journal of Biological Chemistry 263 11486-11492.

Twigg S M & Baxter R C 1998 Insulin-like growth factor (IGF)-binding protein 5 forms an alternative ternary complex with IGFs and the acid-labile subunit. Journal of Biological Chemistry 273 6074-6079.

van Roozendaal C E, Gillis A J, Klijn J G, van Ooijen B, Claassen C J, Eggermont A M, Henzen-Logmans S C, Oosterhuis J W, Foekens J A & Looijenga L H 1998 Loss of imprinting of IGF2 and not H19 in breast cancer, adjacent normal tissue and derived fibroblast cultures. FEBS Letters 437 107-111.

Werner H & LeRoith D 1996 The role of the insulin-like growth factor system in human cancer. Advances in Cancer Research 68 183-223.

Yakar S, Liu J L, Stannard B, Butler A, Accili D, Sauer B & LeRoith D 1999 Normal growth and development in the absence of hepatic insulin-like growth factor I. Proceedings of the National Academy of Sciences of the United States of America 96 7324-7329.

Yamanaka Y, Fowlkes J L, Wilson E M, Rosenfeld R G & Oh Y 1999 Characterization of insulin-like growth factor binding protein-3 (IGFBP-3) binding to human breast cancer cells: kinetics of IGFBP-3 binding and identification of receptor binding domain on the IGFBP-3 molecule. Endocrinology 140 1319-1328.

What is claimed is:

1. A method for treating lung cancer in a patient consisting essentially of administering to said patient a therapeutically effective amount of insulin-like growth factor binding protein-3 (IGFBP-3).

2. The method of claim 1, wherein said therapeutically effective amount of IGFBP-3 is administered parenterally.

3. The method of claim 2, wherein said therapeutically effective amount of IGFBP-3 is administered via subcutaneous injection.

4. The method of claim 2, wherein said therapeutically effective amount of IGFBP-3 is administered intravenously.

5. The method of claim 2, wherein said therapeutically effective amount of IGFBP-3 is administered via intravenous infusion.

6. The method of claim 1, wherein said therapeutically effective amount of IGFBP-3 is about 0.1 mg/kg to 40 mg/kg of body weight.

7. The method of claim 1, wherein said therapeutically effective amount of IGFBP-3 is about 0.25 mg/kg to 5 mg/kg of body weight.

8. The method of claim 1, wherein said IGFBP-3 is nonglycosylated.

9. The method of claim 1, wherein a cytotoxic and/or chemotherapeutic agent is not co-administered with IGFBP-3.
